(19) United States
(12) Patent Application Publication
(10) Pub. No.: US 2007/0282220 A1
(43) Pub. Date: Dec. 6, 2007

(54) DEVICE AND METHOD FOR BUTTON FUSION

(76) Inventor: Dennis L. Abernathie, Columbia, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 11/421,542

(22) Filed: Jun. 1, 2006

(51) Int. Cl. A61B 10/00

(52) U.S. Cl. 600/564

(57) ABSTRACT

The present invention provides a device for cutting and placing a bone button during a button fusion procedure. The device preferably includes a first shaft portion, a second shaft portion rotatably disposed within said first shaft portion, and a cutting portion removably attached to said first shaft portion. When said first shaft portion is rotated, the cutting portion cuts a button of bone, which is then taken into an interior space of said cutting portion. Applying pressure to the second shaft portion forces the button of bone out of the device and into the proper location for a button fusion. The present invention also provides a method of using the device to perform a button fusion.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### BACKGROUND OF THE INVENTION

Spinal fusion, wherein one or more vertebrae of the spine are “fused,” is widely performed for a variety of reasons. For example, spinal fusion may be used to treat broken or fractured vertebra, for correction of spinal deformities such as spinal curves or slippages, for treatment of pain along the spine, for treatment of instability, and in some instances for treatment of cervical disc herniations. Spinal fusions may be used to treat other conditions as well.

Regardless of the condition being treated, in order for a spinal fusion to be successful, a solid union between two or more vertebrae must be achieved. As such, it is important that bone (or a suitable substitute therefor) be used in a spinal fusion procedure. The bone used in the fusion procedure may be taken from a site within the patient's own body, in which case the bone graft is referred to as an autograft, or from a source external to the patient (an allograft). Further, artificial bone substitutes may also be used. Use of an autograft, however, generally results in the most desirable outcome in terms of fusion and healing.

Spinal fusion procedures do, however, pose certain difficulties. Posterior and posterior-lateral fusions, for example, require decortication of facet joints. It is difficult in practice, however, to clean more than approximately 50% of the facet joint. Failure to adequately clean the facet joint and prepare a space for the bone graft increases the likelihood that a spinal fusion procedure will be unsuccessful. Further, matching the size and shape of a bone graft to be delivered is another source of potential error and difficulty in performing a spinal fusion procedure. In addition, when a bone graft is transported from one site on the patient's body to another, care must be taken to maintain the graft within the sterile operating field and not to unnecessarily expose the graft to potential contamination (a problem that is increased if an allograft is used).

What is needed, therefore, is a device and method for cleaning and preparation of a site for spinal fusion, and a device and method for extracting the bone graft, preparing a matched insertion site for the graft, and delivering the graft.

### BRIEF SUMMARY OF THE INVENTION

The present invention provides a device for cutting and placing a bone button during a button fusion procedure. The device preferably includes a first shaft portion, a second shaft portion rotatably disposed within said first shaft portion, and a cutting portion removably attached to said first shaft portion. When said first shaft portion is rotated, the cutting portion cuts a button of bone, which is then taken into an interior space of said cutting portion. Applying pressure to the second shaft portion forces the button of bone out of the device and into the proper location for a button fusion.

In another aspect of the present invention, a spade is included at a first end of the second shaft portion such that tissue on the surface of bone to be cut can be cleared away prior to cutting.

In yet another aspect of the present invention, a piston head is provided attached to the first end of the second shaft portion for pulling a cut button of bone into the device and for inserting the button of bone at a proper location for a button fusion.

In another aspect of the present invention, the device includes a handle attached to, or formed as part of, the first shaft for ease of manipulation of the device and rotation of the first shaft.

In still another aspect of the present invention, the second shaft includes a plurality of indicia along a length thereof for determining the relative positions of the first and second shafts.

In another aspect of the present invention, the first shaft includes a gauge through which a plurality of indicia on the second shaft may be observed.

In another aspect of the present invention, the second shaft further includes a knob portion attached to a second end thereof for ease of manipulation of the second shaft.

In another aspect of the present invention, the cutting portion includes a nut and the first shaft includes a port adapted to receive the nut such that the cutting portion is removably attached to the first shaft.

In another aspect of the present invention, a method is provided for performing a button fusion using the above-described device. During the method, a device according to the present invention is provided, a spade portion is aligned with an area of bone to be cut, the inner shaft of the device is rotated so that the spade can clear away tissue on the surface of the bone, the outer shaft of the device is rotated to cut a button of bone, the device is transferred to the location where the button of bone is to be placed, and pressure is placed on the inner shaft of the device, forcing the button of bone into proper position for performing a button fusion.

In another aspect of the present invention, the button of bone is cut from the spinous process.

### BRIEF DESCRIPTION OF THE DRAWINGS

FIG. 1 is a perspective view of a button fusion device constructed in accordance with the teachings of the present invention.

FIG. 2A is an exploded perspective view of an embodiment of an inner shaft and cutter of a button fusion device constructed in accordance with the teachings of the present invention.

FIG. 2B is an exploded view of an outer shaft and spade of a button fusion device constructed in accordance with the teachings of the present invention.

FIG. 3 is a perspective view of an embodiment of a spade constructed in accordance with the teachings of the present invention.

FIG. 4 is a perspective view of an operative site showing placement of a button within a facet joint in accordance with the teachings of the present invention.

FIG. 5 is a perspective view of an impacted button correctly placed within a facet joint in accordance with the teachings of the present invention.

FIG. 6 is a perspective view of an alternative embodiment of a button fusion device constructed in accordance with the teachings of the present invention.

FIG. 7 is a perspective view of another alternative embodiment of a button fusion device constructed in accordance with the teachings of the present invention.

### DETAILED DESCRIPTION OF THE INVENTION

As used herein, the term “button” refers to an autograft or allograft portion of bone or other suitable material of any size or shape created by a device or method in accordance with the broad teachings of the present invention, or used in conjunction with a device or method in accordance with the broad teachings of the present invention.

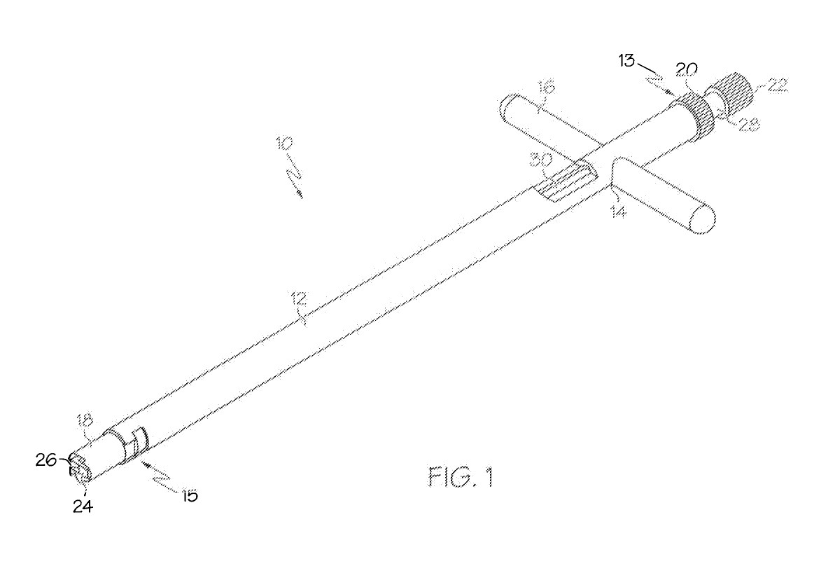

Turning now to the drawings, wherein like numerals indicate like parts, the numeral 10 refers generally to a button fusion device constructed in accordance with the teachings of the present invention. As seen in FIG. 1, button fusion device 10 includes generally an outer shaft 12, a handle 16, a cutter 18, a knob 22, an inner shaft 28, and a spade 24, as well as various other features as described below.

Outer shaft 12 makes up a substantial portion of the body of device 10, defining a longitudinal axis of the device and being adapted to house inner shaft 28 therein. A second end 13 of outer shaft 12 is adapted to receive a nut 20. Second end 13 of outer shaft 12 may be threaded, such that nut 20 is threadably engaged therewith, or may be adapted such that nut 20 may snap into place or may be held in any other suitable fashion. It is preferred that nut 20 is removably engaged by the second end 13 of outer shaft 12. Outer shaft 12 further includes a first end 15, and is substantially hollow along its length such that inner shaft 28 may be inserted in second end 13 of outer shaft 12, extending therethrough and protruding from first end 15 of outer shaft 12.

Outer shaft 12 of button fusion device 10 preferably includes an opening 14 extending therethrough, opening 14 adapted to receive a handle 16 for manipulation of device 10 as described further below. While opening 14 may be located anywhere along the length of outer shaft 12, it is preferred that opening 14 is located nearer second end 13 of shaft 12 than first end 15 of outer shaft 12. In embodiments of the present device wherein opening 14 is located nearer first end 15 of outer shaft 12 than shown in FIG. 1, opening 14 is preferably located at a sufficient distance from first end 15 of outer shaft 12 that handle 16, when inserted through opening 14, has sufficient clearance with respect to a patient's body that device 10 can be used as described below. It is further preferred that opening 14 is positioned such that handle 16 does not obscure the view of the operative site by a user of device 10. It is contemplated that handle 16 may be fixedly attached to outer shaft 12 by insertion into opening 14, however it is preferred that handle 16 is removably attached to outer shaft 12 such that a user of device 10 may insert handle 16 through opening 14 prior to use of device 10, and may thereafter remove handle 16 from opening 14 for easy storage of device 10. In embodiments of the present device wherein handle 16 is fixedly attached to outer shaft 12, it is contemplated that handle 16 and outer shaft 12 may be constructed from a single, unitary piece of material. In embodiments of device 10 utilizing a removably attachable handle 16, handle 16 should include an opening that corresponds to a hollow interior of outer shaft 12 such that inner shaft 28 is able to be inserted through device 10 along a longitudinal axis thereof and is able to pass through handle 16.

Outer shaft 12 of button fusion device 10 preferably further includes a gauge 30 located along the length thereof. Gauge 30 serves to indicate the relative position of inner shaft 28 to outer shaft 12 during use of device 10, as described below. Gauge 30 may be positioned at any location along the length of outer shaft 12, but should be located such that a reading may be taken from gauge 30 during use of device 10, and therefore it is preferred that gauge 30 be located nearer second end 13 of outer shaft 12 than first end 15 of outer shaft 12 so that a reading may be more easily taken therefrom. In the embodiment of device 10 shown in the figures, scoring 32 of inner shaft 28 provides a marking visible although gauge 30 for purposes of measurement of the relative positions of inner shaft 28 and outer shaft 12.

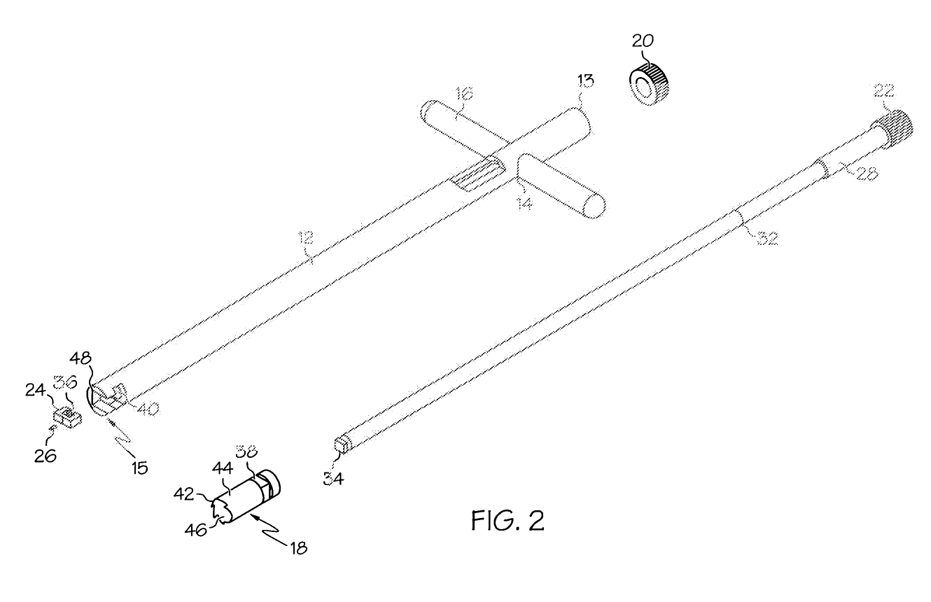

Inner shaft 28 of button fusion device 10 is best shown in FIG. 2A. It is preferred that inner shaft 28 be substantially cylindrical in shape, thus allowing for easy rotation within outer shaft 10. It is contemplated, however, that inner shaft 28 may have any suitable shape and may, for example, have flat edges, forming a substantially square cross-section, so long as the dimensions of inner shaft 28 allow for the free rotation thereof within outer shaft 12. Likewise, although outer shaft 12 is preferably substantially cylindrical in shape, as shown in the figures, it is contemplated that outer shaft 12 may be provided with flat edges or other configurations, and have may any suitable cross-sectional shape. As shown in the figure, inner shaft 28 has a diameter smaller than that of outer shaft 12 so that inner shaft 28 can be inserted through outer shaft 12, extending along the longitudinal axis of outer shaft 12 and protruding from a first end 15 thereof through opening 40. Inner shaft 28 preferably includes scoring 32, which is visible through gauge 30, thereby allowing measurement of the relative positions of inner shaft 28 and outer shaft 12 as necessarily during ordinary use of device 10 as described below. Although inner shaft 28 is shown having scoring 32 at an appropriate location along its length, it is contemplated that scoring 32 may be replaced with any other suitable marker or indicia that can serve to allow a user to measure the relative positions of inner shaft 28 and outer shaft 12, such as, for example, a painted marking or the like.

Inner shaft 28 includes a knob 22 at a second end thereof, such that a user of device 10 can easily rotate inner shaft 28 within outer shaft 12 as described below. Knob 22 is preferably provided with knurls, as shown in FIGS. 1 and 2, or with scoring or texturing, and the like in order to allow a user to easily grasp and turn inner shaft 28 without losing grip on knob 22. Inner shaft 28 further preferably includes a coupling 34 at a first end thereof. Coupling 34 is preferably fixedly attached to inner shaft 28 in such a manner that coupling 34 rotates when inner shaft 28 is rotated by use of knob 22. Coupling 34 is adapted to be removably engaged with an attachment portion 36 of spade 24, as described below.

Cutter 18 includes a nut 38 and a cutting portion 44 with teeth 42. Cutting portion 44 and nut 38 are preferably fixedly attached to one another such that when nut 38 is rotated by means of outer shaft 12, as described below, cutting portion 44 undergoes a corresponding rotation. Teeth 42 are located along an edge of cutting portion 44 of cutter 18, and may be provided in any suitable configuration such that device 10 can be used to cut and remove a portion of bone as described below. It is preferred that cutting portion 44 be constructed from carbonized steel, although any suitable material useful in cutting bone may be used.

In a preferred embodiment of the present device, cutter 18 is removably engaged with outer shaft 12 via the association of nut 38 with port 48 of outer shaft 12, shown in FIGS. 2A and 2B. It is contemplated that various configurations of cutter 18 may be provided, each having, for example, variations in length, width, configuration and arrangement of teeth, and the like. Thus, depending on the specific application for which device 10 is being used, a specific cutter 18 may be utilized. As shown in FIGS. 2A and 2B, nut 38 of cutter 18 is Substantially square shaped and is adapted to be received by port 48 of outer shaft 12. It is contemplated that nut 38 of cutter 18 may have any suitable shape or configuration. Regardless of the shape or configuration of nut 38, however, it is preferred that port 48 of outer shaft 12 is configured to receive and mate with nut 38 such that, when outer shaft 12 is rotated, cutter 18 undergoes a corresponding rotation.

An opening 46 of cutting portion 44 of cutter 18 is adapted to receive a spade 24 therein, spade 24 being configured to receive coupling 34 of inner shaft 28. When spade 24 is attached to coupling 34 of inner shaft 28, inner shaft 28 is locked in place with respect to cutter 18 in terms of motion along a longitudinal axis of device 10, but is freely rotatable within cutter 18. Cutter 18 is correspondingly locked in place via the interaction of nut 38 and port 48 of outer shaft 12, as described above. When the various components of device 10 are engaged with one another, as described, inner shaft 28 is freely rotatable within outer shaft 12 and cutter 18 and may be rotated by a user via rotation of knob 22. When inner shaft 28 is so rotated, spade 24 undergoes a corresponding rotation. Further, when the various components of device 10 are engaged with one another as described, cutter 18 is engaged with port 48 of outer shaft 12 such that when outer shaft 12 is rotated, cutter 18 undergoes a corresponding rotation, moving freely around an outer circumference of spade 24 and rotating independently thereof. Outer shaft 12 is preferably rotated via turning of handle 16 of device 10.

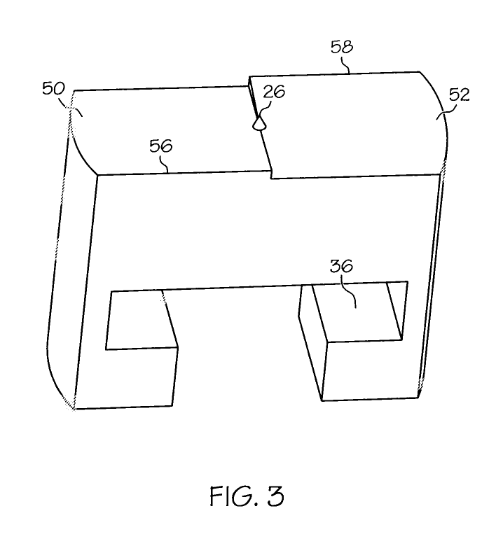

As best seen in FIG. 3, spade 24 preferably includes a point 26. Spade 24 and point 26 are preferably constructed from carbonized steel or other material useful in initiating cutting of bone tissue and the like. Spade 24 may be configured in any suitable manner to provide the cutting functionality (if desired), as described below. As best seen in FIG. 3, spade 24 preferably includes first cutting surface 50 and second cutting surface 52, with each cutting surface disposed such that the sharpened, raised edges 56 and 58 alternately engage a surface to be cut depending on the direction of rotation of spade 24. As also shown in FIG. 3, spade 24 includes an attachment portion 36 adapted to receive coupling 34 of inner shaft 28.

Once each of the various components of device 10 are engaged as described above, device 10 is ready for use in performing a button fusion in accordance with the teachings of the present invention. The device is adapted to remove a portion of bone, thereby creating a ‘button,’ and for delivering the same portion of bone without the need to change instruments during the surgical procedure.

Each of the various components of device 10 described above may be constructed from a variety of suitable materials. It is preferred that portions of device 10 engaged in cutting and the like, such as cutting portion 44 of cutter 18, and spade 24, be constructed from materials such as carbonized steel, stainless steel, and the like in order to provide the required cutting functionality to device 10. Other portions of device 10 may be constructed from other suitable materials, such as, for example, acrylic, PEEK, high-density polyethylene, polypropylene, titanium, aluminum, and the like. With respect to embodiments of the present invention adapted for reuse between various surgical procedures, the materials used for construction of the device should be chosen, in part, for their suitability to sterilization techniques such as autoclaving. With respect to embodiments of the present device intended to be disposable, such devices may be constructed any suitable materials, but it is preferred that such materials are chosen, to the extent possible, for their ability to be recycled or for other reasons associated with the disposability thereof, such as cost.

During use of button fusion device 10, a button or portion of bone is taken from the spinous process, preferably a portion of the iliac crest, of a patient undergoing the button fusion procedure. In taking the portion of bone for use in the button fusion procedure, cutter 18 is first aligned with the spinous process in such a manner as to best extract the required portion of bone, or to allow for extraction of multiple portions of bone if needed. Once cutter 18 is aligned with the spinous process, knob 22 is used to push spade 24 against the spinous process and rotate spade 24 in such a manner as to begin the formation of a hole in the spinous process. In the embodiments of the present invention shown in the figures, point 26 of spade 24 provides a mechanism by which device 10 is maintained in a single position while spade 24 begins formation of the hole and clears away tissue covering the surface of the bone. Without point 26, the hole is less easy to initiate and there is a greater likelihood that device 10 will deviate, even slightly, from its original position once spade 24 is being rotated to begin formation of the hole. Once formation of the hole is begun, the hole is then drilled in the spinous process by rotation of outer shaft 12 of device 10 by use of handle 16. As cutter 18 is rotated along with outer shaft 12, teeth 42 of cutter 18 cut into the spinous process and a portion of bone is taken into cutter 18 due to the offset of teeth 42 along an edge of cutter 18. Retraction of spade 24 during this process allows for the portion of bone to be pulled into cutter 18 of device 10. The thickness of the portion of bone taken may be ascertained by monitoring the movement of scoring 32 of inner shaft 28 within gauge 30. If device 10 is efficiently placed along the spinous process, multiple bone portions or buttons may be taken, each portion of bone being held adjacent one another within device 10. The total thickness of the various bone portions may again be determined by monitoring the movement of scoring 32 of inner shaft 28 within gauge 30.

Although spade 24 is described above as having cutting surfaces 50 and 52 and raised edges 56 and 58 (as shown in FIG. 3), it is contemplated that other structures may be used in place of spade 24. For example, rather than using a spade with a cutting head, a flat, piston-like head portion may be used, the piston head portion begin adapted to removably attach to coupling 34. In such an embodiment, the piston head structure serves no cutting function but rather may be retracted or pushed forward in order to allow space for a button within the present device, or to insert a button into the appropriate opening during a surgical procedure. It is further contemplated that in such an embodiment the edges of the piston head portion may be substantially in contact with an interior wall of cutter 18 such that when the piston head structure is retracted, a vacuum force is created whereby a button is drawn into the device and held in place until insertion into a proper opening during a surgical procedure.

Once one or more buttons have been taken, device 10 is used to prepare a hole at the site in which the button fusion operation is to be performed. It is preferred that device 10 is used directly to create the hole at, for example, a facet joint. This hole is not initiated by spade 24, as described above, because of the presence of one or more portions of bone, or buttons, within device 10 at this stage in the button fusion procedure. This hole is created by engaging cutter 18 with the appropriate location at the site of the facet joint, and rotating outer shaft 12 of device 10 by use of handle 16. This ensures that the hole created at the facet joint has the same dimensions as the hole from which the button was originally obtained. After the hole has been created, decortication of the facet joint results denuded bone on both surfaces of the joint having rough, bony edges. The facet joint is thus in condition to accept one or more buttons.

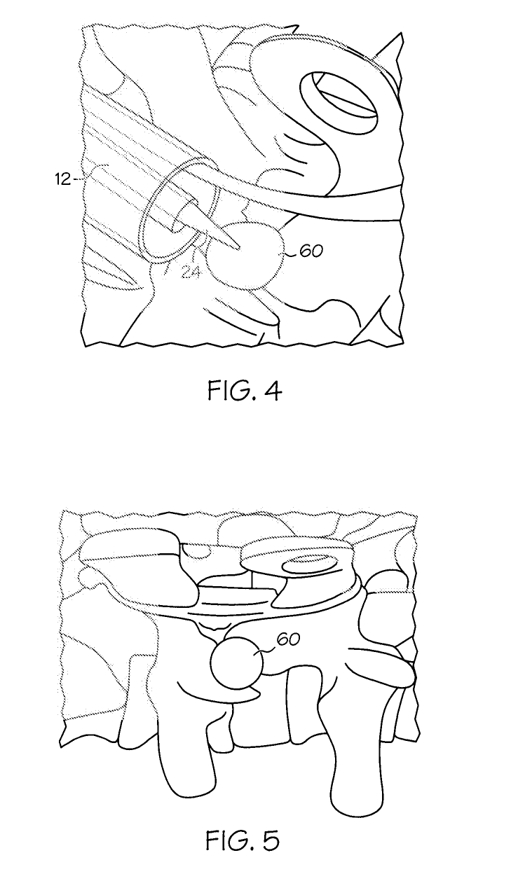

As best shown in FIG. 4, one or more buttons 60 are placed within the newly created hole in the facet joint by use of button fusion device 10. After cutting the new hole as described above, any unwanted material accumulated within cutter 18 of device 10 is expelled therefrom by applying pressure to inner shaft 28, via knob 22, and expelling an appropriate amount of material from the device. Gauge 20 may be used to determine the amount of material expelled from device 10, though it is preferred that visual inspection of device 10 be used to more accurately assess whether the unwanted material has been expelled. Once the unwanted material has been expelled, device 10 is aligned with the newly created hole in the facet joint. One or more buttons 60 contained within device 10 is inserted into the hole created in the facet joint by applying pressure to inner shaft 28 of device 10 as described above. Gauge 30 of device 10 may be used, as described above, to determine how many buttons have been placed within the hole created in the facet joint.

FIG. 5 shows the final position of a button 60 within a hole created in a Facet joint. Once properly placed, button 60 may be held in place by pedicle screws or by a “spider,” or by any other suitable method known in the art.

In another embodiment of this present invention, a button fusion may be performed by extracting a button or portion of bone, as described above, and then by either expelling the button from device 10 so that device 10 can be used to create an opening in which the button bone graft will be placed, or by using another, similarly-sized device 1 0 for creation of the opening. In this embodiment of the present invention, outer shaft 12 is properly aligned at the site in which the opening is to be created. The opening is then created by the rotation of inner shaft 28, which engages spade 24 with the surface of the bone to be cut. Once a suitable opening has been cut, the button previously extracted may be inserted back into device 10 (in the circumstance wherein only one device 10 is being used), or a second device 10 containing the button may be aligned with the newly created opening. The button is then inserted into the openings as described above.

In each of the embodiments above, numerous pre-cut buttons may be used without loss of accuracy in matching the button size with the size of the opening meant to receive the graft. This is true because, although device 10 may be provided in a variety of sizes or shapes, for each size or shape of device 10 a known set of dimensions exist and previously-cut buttons created with the same device, or with a device having similar dimensions, may be utilized in a button fusion procedure. Previously-cut buttons may be obtained from cadavers, artificial bone, or other sources. Use of the present device and method in such a way ensures that there is always a good match in size between the button being used and the opening created to receive the graft.

FIG. 6 provides a perspective view of one alternative embodiment of a button fusion device 110 constructed in accordance with the teachings of the present invention. Shown in the figure are inner shaft 128, gauge 130, scoring 132, outer shaft 112, and cutter 118. These components of device 110 correspond in general principle of operation to the corresponding parts described with respect to FIGS. 1-3, above. Device 110 further includes a dial portion 162. Dial portion 162 serves to adjust the position of inner shaft 128 within outer shaft 112, the relative position of the two shafts being indicated by gauge 130 and scoring 132. Cutter 118 is preferably releasably engaged with device 110 such that various configurations of cutter 118 may be used with the same device 110, each cutter 118 being quickly removed and a new cutter 118 being snapped on or otherwise releasably engaged with device 110. Any suitable method of releasably engaging cutter 118 with device 110 is contemplated as being within the scope of the present invention. Further, device 110 can be used in conjunction with other tools that may be used during various surgical procedures. For example, rather than a cutter 118, device 110 may be provided with an osteotome, curette, awl-tap, or the like. These parts may be readily replaced, allowing for low-cost tools that are consistently sharper than permanent devices that undergo wear over time.

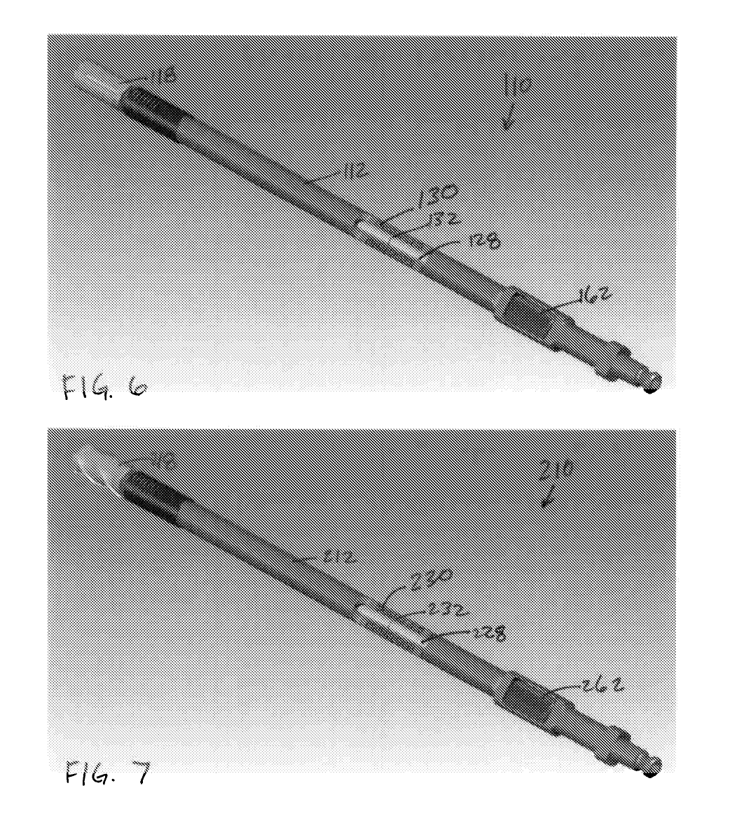

FIG. 7 provides a perspective view of another alternative embodiment of a button fusion device 210 constructed in accordance with the teachings of the present invention. Cutter 218 is provided in one alternative configuration usable with the present device.

The device described above is generally used over a pointed member that is used as a guide. Other aligning methods may also be used, however. For example, it is contemplated that a two-pronged device may be used. One end of such a two pronged device is preferably a round plug that fits within cutter 18, while the other end has two sharp points that are embedded into bone. These points can bridge a boney defect. Rotating the cutter over the cylinder forms a cylindrical cavity that can receive the button plus.

It should be understood that the various descriptions and illustrations of the present device set forth herein are exemplary and are not intended to limit the scope of the present invention. Upon reading this disclosure, many variations and modifications will be apparent to those of skill in the art, and it is contemplated that these variations and modifications are within the spirit and scope of the present invention. For example, variations or modifications in size or configuration of any of the various components of the present device may be made without altering the broad functionality of the invention as described herein. The present invention is not to be limited by size, shape, or configuration, as describe above, or by specific materials recited herein, but is limited only by the claims that follow.

What is claimed is:

1. A device for cutting and placing a bone button during a button fusion procedure, the device comprising:
a first shaft portion, said first shaft portion having a longitudinal axis, a first end, and a second end, said first and second ends disposed at opposite ends of the longitudinal axis of the first shaft portion;
a second shaft portion rotatably disposed within said first shaft portion and along the longitudinal axis thereof, the second shaft portion having a longitudinal axis, a first end, and a second end, said first and second ends disposed at opposite ends of the longitudinal axis of the second shaft portion; and
a cutting portion removably attached to a first end of said first shaft portion, the cutting portion having a substantially cylindrical sidewall defining an interior space thereof, the cutting portion further having a plurality of teeth disposed along an edge of said sidewall,
wherein rotation of said first shaft portion causes a corresponding rotation of said cutting portion.

2. The device according to claim 1 further comprising a spade portion removably attached to a first end of said second shaft portion, wherein rotation of said second shaft portion causes a corresponding rotation of said spade portion, and further wherein longitudinal movement of said second shaft portion causes a corresponding longitudinal movement of said spade portion.

3. The device according to claim 1 further comprising a piston head portion removably attached to a first end of said second shaft portion, wherein rotation of said second shaft portion causes a corresponding rotation of said piston head portion, and further wherein longitudinal movement of said second shaft portion causes a corresponding longitudinal movement of said piston head portion.

4. The device according to claim 1 further comprising at least one handle portion fixedly attached to said first shaft portion such that the device may be more easily grasped and to facilitate rotation of said first shaft portion.

5. The device according to claim 1 further comprising a plurality of indicia along a length of said second shaft portion, whereby a user of the device can determine the relative position of said second shaft portion to said first shaft portion along a longitudinal axis thereof.

6. The device according to claim 5 wherein said first shaft portion further comprises a gauge portion for viewing the relative position of said second shaft portion with respect to said first shaft portion along a longitudinal axis thereof.

7. The device according to claim 1 wherein said second shaft portion further comprises a knob portion fixedly attached thereto at the second end of said second shaft portion to facilitate rotation of said second shaft portion by a user of said device.

8. The device according to claim 1 wherein said cutting portion further comprises a nut fixedly attached thereto, and said first shaft comprises a port at a first end thereof, said port adapted to receive and engage said nut such that rotation of said outer shaft portion causes a corresponding rotation of said cutting portion.

9. The device according to claim 2 wherein said spade portion further comprises a point along an outer surface thereof for properly aligning said spade portion during use of said device.

10. A method of performing a button fusion procedure, the method comprising:
a) providing a device for cutting and placing a bone button during a button fusion procedure, the device comprising:
i) a first shaft portion, said first shaft portion having a longitudinal axis, a first end, and a second end, said first and second ends disposed at opposite ends of the longitudinal axis of the first shaft portion;
ii) a second shaft portion rotatably disposed within said first shaft portion and along the longitudinal axis thereof, the second shaft portion having a longitudinal axis, a first end, and a second end, said first and second ends disposed at opposite ends of the longitudinal axis of the second shaft portion;
iii) a cutting portion removably attached to a first end of said first shaft portion, the cutting portion having a substantially cylindrical sidewall defining an interior space thereof, the cutting portion further having a plurality of teeth disposed along an edge of said sidewall; and
iv) a spade portion removably attached to a first end of said second shaft portion, wherein rotation of said second shaft portion causes a corresponding rotation of said spade portion, and further wherein longitudinal movement of said second shaft portion causes a corresponding longitudinal movement of said spade portion,
wherein rotation of said first shaft portion causes a corresponding rotation of said cutting portion.
b) aligning said spade portion of said device with an area of bone from which a button is to be cut;
c) rotating said inner shaft of said device, thereby causing a corresponding rotation of said spade such that tissue on the surface of said area of bone from which is button is to be cut is removed;
d) rotating said outer shaft of said device such that said cutting portion of said device cuts into said area of bone, said cutting portion cutting a button of bone from said area of bone, said button bone being taken into an interior space of said cutting portion;
e) transferring said device to a location wherein said button of bone is to be inserted to form a button fusion; and
f) exerting pressure on said inner shaft such that said button of bone cut by said cutting portion is pushed into an opening wherein said button of bone is to form a button fusion.

11. The method according to claim 10 wherein the area of bone from which a button is to be cut includes at least a portion of the spinous process.

12. A kit for use with a button fusion device, the button fusion device having a first shaft adapted to removably receive a cutting portion, the kit comprising:
a cutting portion adapted to be removably engaged with the first shaft of a the button fusion device, said cutting portion having a substantially cylindrical sidewall defining an interior space therein;
at least one button portion sized and shaped to be received in the interior space of said cutting portion.

13. The kit according to claim 12 wherein said button portion is comprised of bone.

14. The kit according to claim 13 wherein said button portion further includes an additive for promoting fusion of said bone to at least one bone of a patient undergoing a button fusion procedure.

15. The kit according to claim 14 wherein said additive is bone morphogenic protein.
